(19) United States
(12) Patent Application Publication
(10) Pub. No.: US 2026/0297211 A1
(43) Pub. Date: Oct. 1, 2026

(54) Heterodimeric Antibodies, Uses in Therapeutic Applications, and Compositions Related Thereto

(71) Applicant: Emory University, Atlanta, GA (US)

(72) Inventors: Eric SUNDBERG, Atlanta, GA (US); Jonathan DU, Atlanta, GA (US); Tala AZZAM, Atlanta, GA (US)

(21) Appl. No.: 19/476,326

(22) PCT Filed: Apr. 19, 2024
(86) PCT No.: PCT/US2024/025404

§ 371 Date: Oct. 17, 2025

Related U.S. Application Data
(60) Provisional application No. 63/461,078, filed on Apr. 21, 2023.
(60) Provisional application No. 63/578,789, filed on Aug. 25, 2023.

(51) Int. Cl. C07K 16/30; C07K 16/28

(52) U.S. Cl. CPC C07K 16/30; C07K 16/2809; C07K 16/2812; C07K 16/2815; C07K 16/2818; C07K 16/2866; C07K 2317/31

(57) ABSTRACT

Disclosed herein are mutant heavy chain antibody sequences. In certain embodiments, this disclosure contemplates heterodimeric antibodies with a first heavy chain and a second heavy chain, wherein the first and second heavy chain, or fragments thereof contain different mutations. In certain embodiments, this disclosure relates to nucleic acids and vectors encoding antibodies and recombinant heavy chains disclosed herein and cells containing the same.

### CROSS-REFERENCE TO RELATED APPLICATIONS

This application claims the benefit of U.S. Provisional Application No. 63/461,078 filed Apr. 21, 2023 and U.S. Provisional Application No. 63/578,789 filed Aug. 25, 2023. The entirety of each of these applications is hereby incorporated by reference for all purposes.

### STATEMENT REGARDING FEDERALLY SPONSORED RESEARCH OR DEVELOPMENT

This invention was made with government support under AI149297 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 23010PCT.xml. The XML file is 3 KB, was created on Apr. 19, 2024, and is being submitted electronically via the USPTO patent electronic filing system.

### BACKGROUND

Therapeutic antibodies constitute an important class of drugs for the treatment of a wide range of diseases such as cancer. Their abilities to recruit and stimulate immune system cells, which is often linked to their clinical effectiveness, especially in the immunotherapeutic treatment of cancer, are harbored in the heavy chain constant domains (Fc domains). Fc domains engage Fc receptors and complement in order to induce antibody-mediated effector functions that direct the killing of cells in vivo. Although antibody therapies are successfully used in the treatment of cancer, clinical resistance and refractory disease to these agents continues to be a major issue as recurrence of cancer is not uncommon. Thus, there is a need to identify improvements.
Ridgway et al. report ‘Knobs-into-holes’ engineering of antibody CH3 domains for heavy chain heterodimerization. Protein Engineering, 1996, 9 (7): 617-621.
Ha et al. report an immunoglobulin Fc heterodimer platform. Front Immunol, 2016, 7:394.
Mimura et al. report glycosylation engineering of therapeutic IgG antibodies. Protein Cell 2018, 9 (1): 47-62.
Li et al. report glycosynthase mutants of endoglycosidase S2 show transglycosylation activity. J Biol Chem, 2016, 291 (32): 16508-18.
Moore et al. report a heterodimeric Fc platform engineered for efficient development of bispecific antibodies. Methods, 2019, 154:38-50.
Du et al. report mechanisms and specificities of IgG-active endoglycosidases. Glycobiology, 2020, 30 (4): 268-279.
Klontz et al. report an α-fucosidase for highly efficient IgG transfucosylation. Nat Comm, 2020, 11:620.

References cited herein are not an admission of prior art.

### SUMMARY

This disclosure relates to mutant heavy chain antibody sequences. In certain embodiments, this disclosure contemplates heterodimeric antibodies with a first heavy chain and a second heavy chain, wherein the first and second heavy chain contain different mutations. In certain embodiments, the mutant heavy chain antibody sequences are useful constructing stabilized antibodies useful in therapeutic applications.

In certain embodiments, stabilized heterodimeric antibodies allow for chain selective synthesis or cleavage of glycosylated substituents providing asymmetrically glycosylated antibody constructs.

In certain embodiments, a heterodimeric antibody comprises a first heavy chain having a mutation or combination of mutations disclose herein and a second heavy chain.

In certain embodiments, this disclosure relates to a recombinant antibody heavy chain, or heterodimeric antibody containing the same, with mutations K409S and T411Y, further optionally comprising any of the additional mutations disclosed herein.

In certain embodiments, this disclosure relates to a recombinant antibody heavy chain, or heterodimeric antibody containing the same, with mutations L368S and D399Y, further optionally comprising any of the additional mutations disclosed herein.

In certain embodiments, this disclosure relates to a recombinant antibody heavy chain, or heterodimeric antibody containing the same, with mutations D399Y and K447S, further optionally comprising any of the additional mutations disclosed herein.

In certain embodiments, this disclosure relates to a recombinant antibody heavy chain, or heterodimeric antibody containing the same, with mutations comprising a first heavy chain with mutations K409S and T411Y and a second heavy chain. In certain embodiments, the second heavy chain comprises mutations L368S and D399Y. In certain embodiments, the second heavy chain comprises mutations D399Y and K447S. In certain embodiments, the first heavy chain comprises any of the mutations disclosed herein. In certain embodiments, the second heavy chain comprises any of the mutations disclosed herein.

In certain embodiments, this disclosure relates to a nucleic acid encoding a recombinant heavy chain as disclosed herein in operable combination with a heterologous promoter.

In certain embodiments, this disclosure relates to a vector comprising a nucleic acid encoding the recombinant heavy chain as disclosed herein in operable combination with a heterologous promoter.

In certain embodiments, this disclosure relates to a cell (e.g., somatic cell) comprising a nucleic acid disclosed herein or a vector disclosed herein.

In certain embodiments, this disclosure relates to a non-naturally occurring or heterodimeric antibody comprising a chimeric recombinant antibody heavy chain(s) as disclosed herein.

In certain embodiments, the first heavy chain of the antibody comprises mutations K409S and T411Y. In certain embodiments, the second heavy chain of the antibody comprises mutations L368S and D399Y.

In certain embodiments, the first heavy chain of the heterodimeric antibody comprises mutations K409S and T411Y. In certain embodiments, a second heavy chain of the antibody comprises mutations D399Y and K447S.

### BRIEF DESCRIPTION OF THE SEVERAL VIEWS OF THE DRAWINGS

FIG. 1 shows a diagram of the potential constructs formed upon co-expression of the heavy chain, light chain and Fc plasmids. Relative increased percentage formation of the Fab1Fc heterodimer using WT rituximab sequences, subsequently detected by MS, were used to determine stability of specific mutations in comb YSelect 1 and comb YSelect 2.

FIG. 2A shows data for constructs with comb YSelect 1 mutations. Histograms indicate the relative percentage of heterodimerization as determined by intact LC/MS when some or all Protomer A mutations are on the fragment containing a Fab and Fc and Protomer B mutations are on the fragment containing only an Fc (right) or when the orientation is reversed (left).

FIG. 2B shows data for constructs with comb YSelect 2 mutations. Histograms indicate the relative percentage of heterodimerization as determined by intact LC/MS when some or all Protomer A mutations are on the fragment containing a Fab and Fc and Protomer C mutations are on the fragment containing only an Fc (right) or when the orientation is reversed (left).

FIG. 3A illustrates bridging assay using a bispecific antibody in which the Fc of Protomer A (combYSelect 1 & 2) is engineered with an anti-CD20 rituximab Fab and the Fc Protomer B (combYSelect 1) or Protomer C (combYSelect 2) is attached to an anti-HER2 nanobody. Binding to CFSE stained Raji cells and Calcein-Violet BT474 cells is assessed.

FIG. 3B shows data from flow cytometry density plots and a scatter plot depicting cell cluster formations of Raji and BT474 cells when combYSelect 1, 2, a non-specific IgG1 isotype control, or no antibody was added to the cell mixture. Each comb YSelect IgG was compared to both the no antibody and isotype control.

FIG. 3C illustrates a combYSelect 2 cytokine trap and the luciferase inhibition cell-based assay.

FIG. 3D shows data on the percentage of luciferase response normalized to that when no inhibitor is added which is plotted as a function of varying inhibitor concentration in the presence of 5 pM of IL1β. The IC₅₀ values were determined using a non-linear least square fit.

### DETAILED DESCRIPTION

Before the present disclosure is described in greater detail, it is to be understood that this disclosure is not limited to embodiments described, and as such may, of course, vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, have the meaning ascribed to them in U.S. Patent law in that when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

As used herein, an “oxazoline” refers a chemical group with a 5-(hydroxymethyl)-2-methyl-3a,6,7,7a-tetrahydro-5H-pyrano[3,2-d]oxazole-6,7-diol structure optionally substituted with a glycol, saccharides, or polysaccharide, which can be prepared by the procedures set out in Noguchi et al. J. Org. Chem. 2009, 74, 2210-2212. Reactions with glycols, saccharides and polysaccharide results in N-acetyl-2-amino sugars when catalyzed by the EndoS2 D184M enzyme.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen, and thereby targeting the antigen for degradation or elimination, or any cell or organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibody, or non-naturally produced synthetic antibodies, such as specific binding single chain antibodies, bispecific antibodies, or fragments thereof. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence; however, embodiments of this disclosure are directed to antibodies that have two heavy chains that are dissimilar sequences or have alternative mutation patterns. Similarly, the two light chains typically have the same amino acid sequence; however, light chains with dissimilar sequences are contemplated. Often each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

A “chimeric antibody” is a molecule in which different portions of the antibody are derived from different immunoglobulin molecules such that the entire molecule is not naturally occurring. Examples of chimeric antibodies include those having a variable region derived from a non-human antibody and a human immunoglobulin constant region. The term is also intended to include antibodies having a variable region derived from one human antibody grafted to an immunoglobulin constant region of a predetermined sequences or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. See Jefferis et al. mAb, 1 (2009), pp. 332-338. In certain embodiments, the antibody, antibody heavy chain, antigen binding fragment, the light chain, or the heavy chain comprises a non-naturally occurring chimeric amino acid sequence such that there is at least one mutation that is not present in naturally occurring antibodies.

Smith et al. report a protocol for the production of antigen-specific chimeric human monoclonal antibodies (hmAbs) wherein antibody-secreting cells (ASCs) are isolated from whole blood collected after vaccination and sorted by flow cytometry into single cell plates. Nat Protoc. 2009; 4 (3): 372-84. The antibody genes of the ASCs are then amplified by RT-PCR and nested PCR, cloned into expression vectors and transfected into a human cell line. Meijer et al. report methods for isolation of human antibody repertoires with preservation of the natural heavy and light chain pairing. J Mol Biol, 2006, 358 (3): 764-72. Wrammert et al. report using immunoglobulin variable regions isolated from sorted single ASCs to produce human monoclonal antibodies (mAbs) that bound with high affinity. Nature, 2008, 453 (7195): 667-671.

Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28 (4/5): 489-498; Studnicka et al., 1994, Protein Engineering 7:805; and Roguska et al., 1994, Proc. Natl. Acad. Sci. USA 91:969), and chain shuffling (U.S. Pat. No. 5,565,332).

Polypeptides sequences of the heavy chains and antibodies containing the same, as disclosed herein, can be produced by any commonly used method. Typical examples include the recombinant expression in suitable host systems, e.g., cell, mammalian cell, bacteria, or yeast. In general, the polypeptides may be produced by living host cells that have been genetically engineered to produce the polypeptide. Methods of genetically engineering cells to produce proteins are well known in the art. See e.g., Ausubel et al., eds. (1990), Current Protocols in Molecular Biology (Wiley, New York). Such methods include introducing nucleic acids that encode and allow expression of the polypeptide into host cells. These host cells can be bacterial cells, fungal cells, or animal cells grown in culture. In one embodiment, polypeptides are produced in mammalian cells. Typical mammalian host cells for expressing the peptide include Chinese Hamster Ovary (CHO cells), lymphocytic cell lines, e.g., NSO myeloma cells, SP2 cells, COS cells.

In addition to the nucleic acid sequences encoding the peptide, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). For example, typically the selectable marker gene confers resistance to drugs, such as G418, hygromycin, or methotrexate, on a host cell into which the vector has been introduced.

Standard molecular biology techniques can be used to prepare the recombinant expression vector, transfect the host cells, select for transformants, culture the host cells and recover the peptides or cells coated with the peptide from the culture medium. For example, the peptides or cells can be isolated by affinity chromatography.

In certain embodiments, this disclosure relates to nucleotide sequences or nucleic acids that encode the heavy chain peptides, or antibody containing the same, as disclosed herein, genetic constructs that include nucleotide sequences or nucleic acids and one or more elements for genetic constructs known per se. In certain embodiments, this disclosure relates to hosts or host cells that contain such nucleotide sequences or nucleic acids, and/or that express (or are capable of expressing), the heavy chain peptides disclosed herein.

In certain embodiments, this disclosure relates to methods for preparing heavy chain peptides or antibodies containing the same or cells expressing the heavy chain peptide or antibodies containing the same using constructs disclosed herein, which method comprises cultivating or maintaining a host cell under conditions such that said host cell produces or expresses the heavy chain peptide(s) or antibody constructs thereof as disclosed herein.

As used herein, a “mutation,” “mutant,” or the like of an antibody heavy chain sequence refers to the expression of a variant amino acid(s) within a heavy chain antibody defined by positions compared to base amino acids within the sequence segment, e.g., of UNIPROTKB/SWISS-PROT having accession number P01857.1.

STKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPE LLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVY TLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPGK (SEQ ID NO: 1), wherein the N-terminal amino acid serine(S) is position 119. The mutants may be constructed by building peptide sequences synthetically or, more typically, constructed using recombinant nucleic acid techniques, e.g., expression of the heavy chain in a cell from a template nucleic acid. Due to three codon translation of amino acids from nucleic acid, several three nucleotide codons may express the same amino acid variant. Sometimes the variant is due to a single nucleotide change, and sometimes the variant is due to more than one nucleotide change. Thus, reference to a “mutation,” “mutant,” or the like of a heavy chain antibody sequence are not necessarily limited to solely single nucleotide changes.

As used herein, the term “cell” refers to a biological compartment containing a lipid membrane and cytosol which may contain a nucleus containing genetic material, mitochondria, and other organelles. In certain embodiments, the cell is a somatic cell.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acids joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free expression system. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a heavy chain peptide(s) or antibodies containing the same as disclosed herein in operable combination with a heterologous promoter.

In certain embodiments, this disclosure contemplates a heavy chain peptide or antibody containing the same, as reported herein, conjugated to a label. A “label” refers to a detectable moiety that is conjugated directly or indirectly to another molecule, such as an antibody or a protein, to facilitate detection of that molecule. Specific, non-limiting examples of labels include fluorescent tags, enzymatic linkages, and radioactive isotopes. A label includes the incorporation of a radiolabeled amino acid or the covalent attachment of biotinyl moieties to a peptide that can be detected by marked avidin (for example, streptavidin containing a fluorescent marker or enzymatic activity that can be detected by optical or colorimetric methods). Various methods of labeling peptides and glycoproteins are known in the art and may be used. Examples of labels for peptides include, but are not limited to, the following: radioisotopes or radionucleotides (such as ¹⁸F, ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined peptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms (linking groups) of various lengths to reduce potential steric hindrance.

In certain embodiments, the disclosure relates to recombinant antibody heavy chain peptides and antibodies containing the same, comprising sequences disclosed herein or variants or fusions thereof wherein the interior amino acid sequence, the amino terminal end, or the carbon terminal end of the amino acid sequence are optionally attached to a heterologous amino acid sequence, label, or reporter molecule.

In certain embodiments, the disclosure relates to the recombinant vectors comprising a nucleic acid encoding an antibody heavy chain peptide or antibody containing the same as disclosed herein. In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env. In certain embodiments, the recombinant vector optionally comprises a gene vector element (nucleic acid) such as a selectable marker region, lac operon, a CMV promoter, a hybrid chicken β-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

In certain embodiments, antibody heavy chain peptides or antibodies containing the same are conjugated to a therapeutic agent or cytotoxic agent for uses in treating disease or conditions such as cancer. The term “cytotoxic agent” as used herein refers to a substance that inhibits or prevents the function of cells and/or causes destruction of cells. The term is intended to include radioactive isotopes, chemotherapeutic agents e.g., methotrexate, adriamycin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents, or growth inhibiting agent. A “growth inhibitory agent” when used herein refers to a compound or composition which inhibits growth of a cell, either in vitro or in vivo. Examples of growth inhibitory agents include agents that block cell cycle progression, such as agents that induce G1 arrest and M-phase arrest. Such inhibitory agents include, for example, the vinca alkaloids (vincristine, vinorelbine and vinblastine), paclitaxel, and topoisomerase II inhibitors such as doxorubicin, epirubicin, daunorubicin, etoposide, bleomycin, tamoxifen, prednisone, dacarbazine, mechlorethamine, cisplatin, methotrexate, 5-fluorouracil, and cytarabine.

In certain embodiments, this disclosure contemplates antibody heavy chains or antibodies containing the same, wherein a desired sugar chain is added to a core fucosylated or nonfucosylated GlcNAc-acceptor, including fucosylated or nonfucosylated GlcNAc-IgG acceptor. As such, the present disclosure allows for the synthesis and remodeling of therapeutic antibodies, or Fc fragments thereof, to provide for certain biological activities, such as, prolonged half-life time in vivo, less immunogenicity, enhanced in vivo activity, increased targeting ability, and/or ability to deliver a therapeutic agent.

### Antibody Heavy Chains, Glycosylations, and Mutations

In certain embodiments, a recombinant antibody heavy chain comprises a first heavy chain having an of a mutation or combination of mutations disclose herein and a second heavy chain.

In certain embodiments, this disclosure relates to a recombinant antibody heavy chain, or heterodimeric antibody containing the same, with mutations K409S and T411Y, further optionally comprising any of the additional mutations disclosed herein.

In certain embodiments, this disclosure relates to a recombinant antibody heavy chain, or heterodimeric antibody containing the same, with mutations L368S and D399Y, further optionally comprising any of the additional mutations disclosed herein.

In certain embodiments, this disclosure relates to a recombinant antibody heavy chain, or heterodimeric antibody containing the same, with mutations D399Y and K447S, further optionally comprising any of the additional mutations disclosed herein.

In certain embodiments, this disclosure relates to a recombinant antibody heavy chain, or heterodimeric antibody containing the same, with mutations comprising a first heavy chain with mutations K409S and T411Y and a second heavy chain. In certain embodiments, the second heavy chain comprising mutations L368S and D399Y. In certain embodiments, the second heavy chain comprising mutations D399Y and K447S. In certain embodiments, the first heavy chain comprises any of the mutations disclosed herein. In certain embodiments, the second heavy chain comprises any of the mutations disclosed herein.

In certain embodiments, this disclosure relates to a nucleic acid encoding the recombinant heavy chain as disclosed herein in operable combination with a heterologous promoter.

In certain embodiments, this disclosure relates to a vector comprising a nucleic acid encoding the recombinant heavy chain as disclosed herein in operable combination with a heterologous promoter.

In certain embodiments, this disclosure relates to a cell (e.g., somatic cell) comprising a nucleic acid disclosed herein or a vector disclosed herein.

In certain embodiments, this disclosure relates to a non-naturally occurring or chimeric heterodimeric antibody comprising a recombinant antibody heavy chain(s) as disclosed herein.

In certain embodiments, the non-naturally occurring or chimeric heterodimeric antibody recombinant antibody or first heavy chain is conjugated to a first glycan at Asn297 (N297) and the second heavy chain is conjugated to a second glycan at Asn297 (N297). In certain embodiments, the first and second glycan are the same glycan or are not the same glycan.

In certain embodiments, the non-naturally occurring or chimeric heterodimeric antibody or first heavy chain is conjugated to a first glycan at Asn297 (N297), and the second heavy chain is conjugated to a second glycan at Asn297 (N297). In certain embodiments, the first and second glycan are the same glycan or are not the same glycan. In certain embodiments, the first glycan and the second glycan are the same glycan. In certain embodiments, the heterodimeric asymmetrically glycosylated antibody contains at least one N-acetylglucose amine (GlcNAc) or fucosylated N-acetylglucosamine (Fucal, 6GlcNAc), e.g., at N297.

In certain embodiments, this disclosure contemplates a single chain antibody having one or more of the mutations disclosed herein.

In certain embodiments the mutations are variants. In certain embodiments, the variant comprises conservative amino acid substitutions. In certain embodiments, the variant comprises nonconservative amino acid substitutions. In certain embodiments, the conservative and/or nonconservative amino acid substitutions are within the framework regions. In certain embodiments, the variant does not contain substitutions within the light chain CDR1, CDR2, or CDR3.

It is noted that in reference to SEQ ID NO: 1, it is specifically for IgG1. IgG2, IgG3, and IgG4 will have some alternative amino acids at the same positions. For example, IgG2 contains a V at position 309 instead of L at position 309 for IgG1. IgG4 contains a F at position 234 instead of L at position 234 for IgG1.

In certain embodiments, this disclosure relates to antibodies reported wherein the constant region comprises a mutation that activates immune responses, enhance ADC by increasing FcγRIIIa binding or decreasing FcγRIIb binding, enhance ADCP by increasing FcγRIIa binding or increased FcγRIIIa binding, enhance CDC by increasing C1q binding or hexamerization, reduce effector functions by aglycosylation, reducing FcγR and C1q binding, increasing co-engagement by increasing FcγRIIb binding, increasing FcγRIIa binding, or decreasing FcγRIIIa binding, and/or increases half-life. Examples of these mutations are further provided below.

In certain embodiments, this disclosure contemplates that a heavy chain contains at least one mutation wherein both heavy chains are not identical. In certain embodiments, this disclosure contemplates that one heavy chain may have alternative mutations than the opposite heavy chain, i.e., one of the two heavy chains contain a mutation that the other sequence does not, or one of the two heavy chains contain one or more mutations, and the other heavy chain contains different mutations.

### Antibodies, Bispecific Antibodies, and Further Mutations

In certain embodiments, the heterodimer heavy chains comprising mutations disclosed herein are used to produce antibodies or bispecific antibodies with binding specificity for therapeutic targets. In certain embodiments, the heavy and light chain variable sequences are combined with, grafted, or joined with the heave chain mutations, constant regions/sequences disclosed herein. In certain embodiments, a first heavy-light chain pair having a first specificity and a second heavy-light chain pair having a second specificity different from the first specificity are produced separately and then mixed for heavy chain-heavy chain pairing having mutations/combinations of mutations disclosed herein.

In certain embodiments, the heterodimeric antibody comprises recombinant antibody heavy chain(s) as disclosed herein.

In certain embodiments, the antibody heavy chain with mutations disclosed herein preferentially forms a heterodimer with a second heavy chain having mutants wherein the second heavy chain comprises a second set of mutations not in the first chain.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y.

In certain embodiments, the first or second heavy chain further has one, two, three, four, five, six, seven, eight, nine, ten, eleven, twelve, thirteen, fourteen, fifteen or more of the following mutations G236A, S239D, A330L, I332E, S267E, L328F, P238D, H268F, S324T, S228P, G236R, L328R, L234A, L235A, M252Y, S254T, T256E, M428L, N434S, P329G, D265A, N297A, N297G, N297Q, F243L, R292P, Y300L, V305I, P396L, S298A, E333A, K334A, L234Y, L235Q, G236W, S239M, H268D, D270E, K326D, A330M, K334E, K326W, E333S, E345R, E430G, S440Y, L235E, N325S, wherein the mutation is in reference to positions in amino acid sequence (SEQ ID NO: 1) (segment of UNIPROTKB/SWISS-PROT: P01857.1), wherein the N-terminal amino acid serine(S) is position 119.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations L234A or L235A, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations L234A, L235A, or P329G, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations D265A or N297A, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations D265A or N297G, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations D265A or N297Q, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations M252Y, S254T, or T256E, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations F243L, R292P, Y300L, V305I, or P396L, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S239D or I332E, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S239D, I332E, or A330L, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S239D, I332E, G236A, or A330L, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S298A, E333A, or K334A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S298A, E333A, or K334A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations L234Y, L235Q, G236W, S239M, H268D, D270E, or S298A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations D270E, K326D, A330M, or K334E, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations G236A, S239D, or I332E, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations K326W or E333S, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations E345R, E430G, or S440Y, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations E345R, E430G, or S440Y, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S267E or L328F, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S267E, L328F, or P238D, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations N325S or L328F, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations S267E, H268F, or S324T, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations M428L or N434S, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations L368S and D399Y. In certain embodiments, the first or second heavy chain further has mutations M252Y, S254T, T256E, M428L, or N434S, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations I253A, H310A, Q311A, N315A, E430A, or H435A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has one, two, three, four, five, six, seven, eight, nine, ten, eleven, twelve, thirteen, fourteen, fifteen or more of the following mutations G236A, S239D, A330L, I332E, S267E, L328F, P238D, H268F, S324T, S228P, G236R, L328R, L234A, L235A, M252Y, S254T, T256E, M428L, N434S, P329G, D265A, N297A, N297G, N297Q, F243L, R292P, Y300L, V305I, P396L, S298A, E333A, K334A, L234Y, L235Q, G236W, S239M, H268D, D270E, K326D, A330M, K334E, K326W, E333S, E345R, E430G, S440Y, L235E, N325S, wherein the mutation is in reference to positions in amino acid sequence (SEQ ID NO: 1) (segment of UNIPROTKB/SWISS-PROT: P01857.1), wherein the N-terminal amino acid serine(S) is position 119.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations L234A or L235A, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations L234A, L235A, or P329G, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations D265A or N297A, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations D265A or N297G, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations D265A or N297Q, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations M252Y, S254T, or T256E, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations F243L, R292P, Y300L, V305I, or P396L, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S239D or I332E, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S239D, I332E, or A330L, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S239D, I332E, G236A, or A330L, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S298A, E333A, or K334A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S298A, E333A, or K334A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations L234Y, L235Q, G236W, S239M, H268D, D270E, or S298A, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations D270E, K326D, A330M, or K334E, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations G236A, S239D, or I332E, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations K326W or E333S, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations E345R, E430G, or S440Y, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations E345R, E430G, or S440Y, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S267E or L328F, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S267E, L328F, or P238D, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations N325S or L328F, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations S267E, H268F, or S324T, or all, or combinations thereof.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations M428L or N434S, or both.

In certain embodiments, this disclosure relates to a heterodimeric antibody heavy chain, or antibody containing the same, with mutations comprising a first heavy chain with mutations K409S and T411Y and a second heavy chain. In certain embodiments, the second heavy chain comprising mutations D399Y and K447S. In certain embodiments, the first or second heavy chain further has mutations M252Y, S254T, T256E, or M428L, N434S or all, or combinations thereof.

### Heterodimeric IgG Molecules

Mutations have been made in each of the two IgG heavy chain protomers to promote the pairing of distinct like protomers, thereby resulting in heterodimerization of the IgG heavy chains. IgG heterodimers may be use therapeutically, e.g., in bi-specific antibodies that include two distinct antibody-binding fragment (Fab) molecules that recognize distinct antigens.

Mutations to produce heterodimers of the IgG/Fc interface were investigated. An in silico approach was used where residues in the CH3-CH3 interface of IgG were mutated to either tyrosine (Y) or serine(S) and the change in binding energy (ΔΔG) was estimated using the ‘interface’ mode of Rosetta 2.3. Residues in the CH3 region which were within 4 angstroms of the opposing CH3 domain were selected for the screening. A total of 31 residues were found: 347Q, 349Y, 350T, 351L, 352P, 353P, 354S, 355R, 356D, 357E, 360K, 364S, 366T, 368L, 370K, 390N, 392K, 393T, 394T, 395P, 397V, 398L, 399D, 400S, 405F, 406L, 407Y, 409K, 411T, 444S and 447K. The in silico screen was carried out by introducing up to two mutations in each chain.

### Evaluation of IgG Heterodimer Formation

The results were filtered for scores where the ΔΔG of the heterodimer was less than or equal to −0.8 kcal/mol and the ΔΔG of the homodimers were equal to or greater than 0 kcal/mol. From this selection, two sets of mutations were chosen for experimental evaluation. In order to evaluate the efficacy of these mutations on heterodimer formation, the mutations were introduced into the heavy chain (Hc) and Fc constructs of rituximab. The constructs were then co-transfected into HEK293F cells along with the light chain (Lc) of rituximab. In addition to the mutants that were identified, the mutations for the knobs-in-holes and electrostatic steering strategies were also tested.

The mutations are defined by positions compared to base amino acids within the sequence segment of UNIPROTKB/SWISS-PROT having accession number P01857.1. STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR DELTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 1), wherein the N-terminal amino acid serine(S) is position 119.

IgG Heterodimers Exhibit Improved Heterodimerization Rates without Impacting IgG Stability or Function

The heterodimers comb YSelect 1 IgGs (L368S/D399Y-K409S/T411Y) and combYSelect 2 IgGs (D399Y/K447S-K409S/T411Y) were engineered into a bispecific antibody and a cytokine trap for evaluating therapeutic applications. Heterodimers L368S/D399Y-K409S/T411Y, D399Y/T411Y-K409S/T411Y and D399Y/K447S-K409S/T411Y were identified as having desirable stability by computer modeling methods.

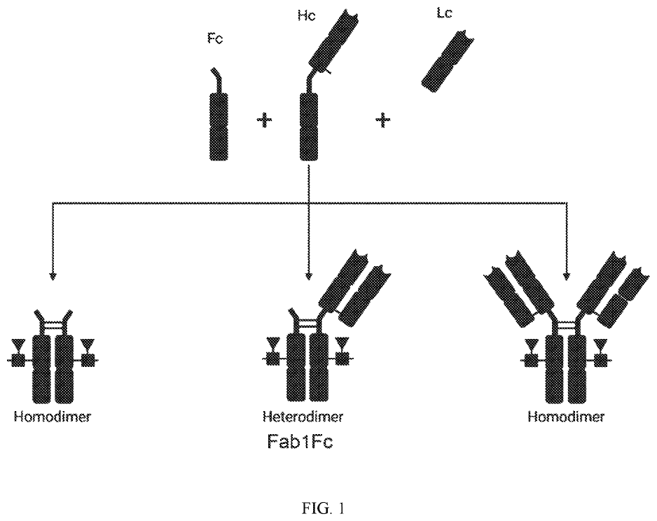

A model system was developed involving the coexpression of an IgG1 Fc region and full-length heavy chain (Hc) of rituximab, each from distinct protomers in a heterodimer design, as well as the light chain (Lc) of rituximab, as a model IgG1 antibody. After purification by protein A affinity chromatography and treatment with the IgG-specific endoglycosidase EndoS2 to remove heterogeneous glycosylation at residue Asn297, three potential products with unique masses that are detectable by liquid chromatography-mass spectrometry (LC-MS) due to coexpression including: an intact Fc region (about 50 kDa), an intact IgG (about 150 kDa), and a monovalent IgG molecule (Fab1Fc; about 100 kDa) (FIG. 1). The formation of intact Fc regions and IgG antibodies each result from the two possible homodimeric pairs, while the formation of the Fab1Fc molecule arises strictly from heterodimerization. Thus, the ratio of Fab1Fc to Fc and IgG is a direct measure of heterodimer formation.

Rituximab was evaluated in this assay. Fab1Fc at 63 percent was observed. For the knob-in-hole (KiH) heterodimer (T366Y-Y407T) i.e. Ridgway et al. 1996, heterodimerization was not detected when the Hc, containing the T366Y mutation, was co-expressed with the Fc containing the Y407T mutation, along with the light chain. However, 57% Fab1Fc formation was observed when the Y407T mutation was on the heavy chain and the T366Y mutation was on the Fc. The electrostatic steering heterodimer that was tested resulted in 91% Fab1Fc formation regardless of which chain contained each set of mutations. In comparison to the controls above, 97% Fab1Fc formation was observed for both mutation-chain combinations for combYSelect 1 having L368S/D399Y-K409S/T411Y. Similarly, combYSelect 2 D399Y/K447S-K409S/T411Y resulted in 96% Fab1Fc formation when the D399Y/K447S mutations were contained within the Hc and 91% when that set of mutations was contained in the Fc. These results indicate that the heterodimers exceed those of the other previously reported heterodimers that were tested.

TABLE 1
provides a summary of the experimental results

| Chain A | Chain B | % Heterodimer | |
|---|---|---|---|
| mutations | Mutations | formation | Mutation |
| T366Y | Y407T | 29.0 | Knobs-in-holes - Ridgway et al. 1996 |
| E357K/S364K | L368D/K370S | 91.0 | Electrostatic steering- Moore et al. 2019 |
| L368S/D399Y | K409S/T411Y | 97.1 | New mutations in this disclosure |
| D399Y/K447S | K409S/T411Y | 93.6 | |

### Heterodimer Mutations do not Impact IgG Stability or FcRn Binding

In addition to promoting heterodimer formation, experiments were performed to evaluate whether the mutations introduced in the CH3 domains affect the functional properties of IgG molecules. The thermal stability of each construct as assessed by determining the melting curves of the resulting heterodimeric antibodies in the Fab1Fc form (i.e., the product resulting from co-expression of the heavy chain, light chain, and Fc fragment). Rituximab had three transition temperatures: 74.9° C., 81.8° C., and 90° C. corresponding to the unfolding of the CH2, Fab and CH3 domains respectively. Additionally, the near identical transition temperatures between the rituximab IgG and rituximab in the Fab1Fc form confirmed that thermal stability of the latter is representative of the full-length IgG. In contrast, the knob-in-hole and electrostatic steering constructs, all of which are in the Fab1Fc form, only showed two transition temperatures corresponding to the CH2 and Fab domains. The first transition temperature ranged from 72.2° C. to 74.8° C., whereas the second was between 80.8° C. and 81.7° C. When we assessed the melting temperatures of combYSelect 1 and 2 Fcs, only one melting temperature at approximately 74° C. was observed, which usually corresponds to the CH2 domain. This suggests that the CH3 domain of the heterodimers either unfolds at the same temperature as the CH2 domain or only undergoes reversible unfolding within our tested temperature range.

The potential effects of these mutations on the overall structure of the CH2-CH3 interface was also assessed. This region is of particular interest as it is where the neonatal Fc receptor (FcRn) binds in a pH-dependent manner to recycle endogenous IgGs and extend their serum half-life. Biolayer interferometry (BLI) analysis was used to compare the FcRn binding affinity of rituximab to comb YSelect 1 and 2 IgGs with rituximab Fab domains. KD values of 14.3, 17.0 and 8.0 nM were obtained for rituximab, combYSelect1 and comb YSelect2, respectively, at pH 6. These IgGs showed minimal binding to FcRn at pH 7.4.

### Mutations for Optimal Heterodimerization

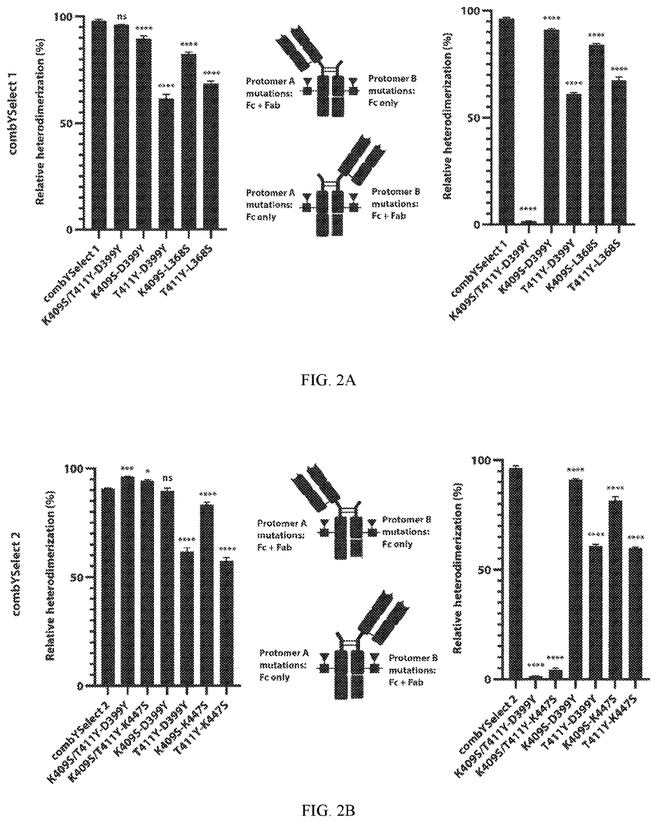

An LC/MS method for assessing heterodimerization was used to determine whether mutations are essential for optimal heterodimerization. The L368S mutation was dispensable when the D399Y mutation was on an Fc, with protomer A mutations on the heavy chain. The observed 96% heterodimerization for K409S/T411Y-D399Y was not significantly different from comb YSelect 1. However, there was no detectable heterodimerization when the D399Y mutation was on the Fc fragment attached to the Fab, suggesting that L368S might play a role in stabilization of the heavy chain (FIG. 2A). On the other hand, 96% and 94% Fab1Fc formation for the K409S/T411Y-D399Y and K409S/T411Y-K447S Hc-Fc constructs were observed respectively, which indicated improved heterodimerization compared to comb YSelect 2. However, when the D399Y and K447S individual mutations were contained in the heavy chain, they were required for heterodimer formation (FIG. 2B). Finally, constructs with single mutations on each chain in all 4 possible combinations: K409S-D399Y, T411Y-D399Y, K409S-L368S, and T411Y-L368S for combYSelect1 and K409S-D399Y, T411Y-D399Y, K409S-K447S, and T411Y-K447S for comb YSelect2 have significantly reduced heterodimerization regardless of mutation-chain pairing (FIG. 2A-2B).

IgG1 Heterodimers Predicted by combYSelect have Therapeutic Applications

IgG heterodimers are often used for the development of bispecific monoclonal antibodies and Fc fusion proteins that exhibit unique and/or improved therapeutic properties. To confirm that combYSelect heterodimers can be utilized broadly for potential therapeutic applications, combYSelect-based bispecific antibodies and cytokine traps were engineered and tested their abilities to bind simultaneously to two target cell lines expressing distinct antigens and to inhibit cytokine-mediated cellular signaling, respectively.

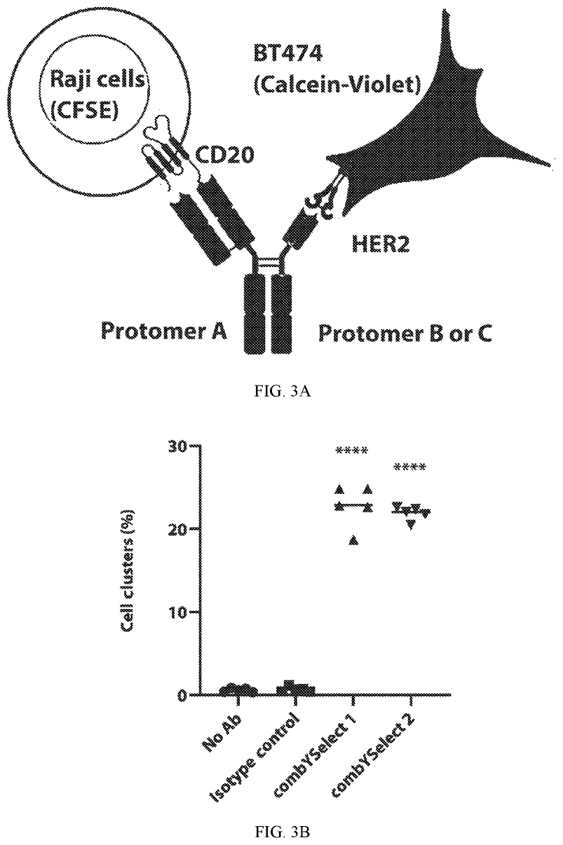

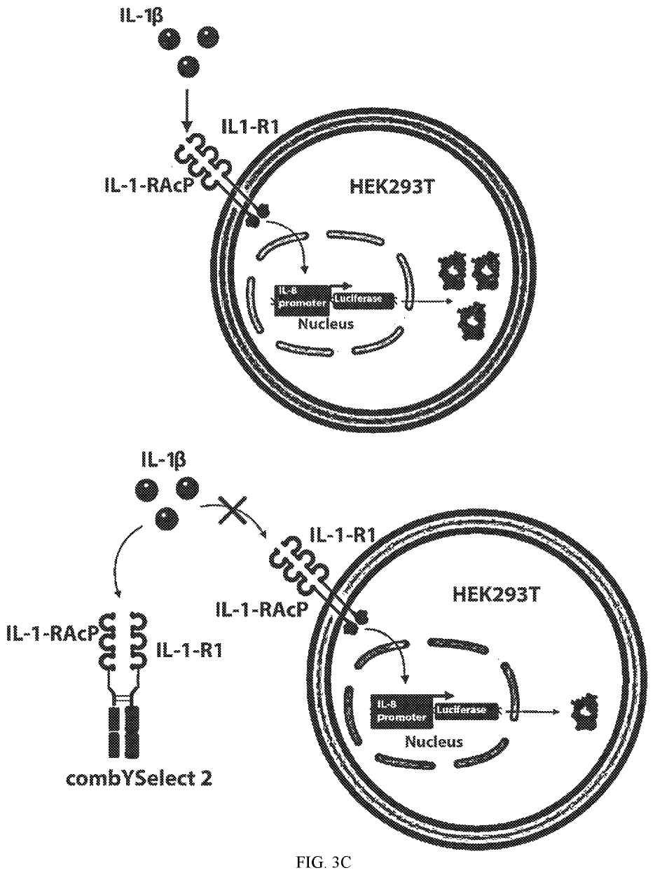

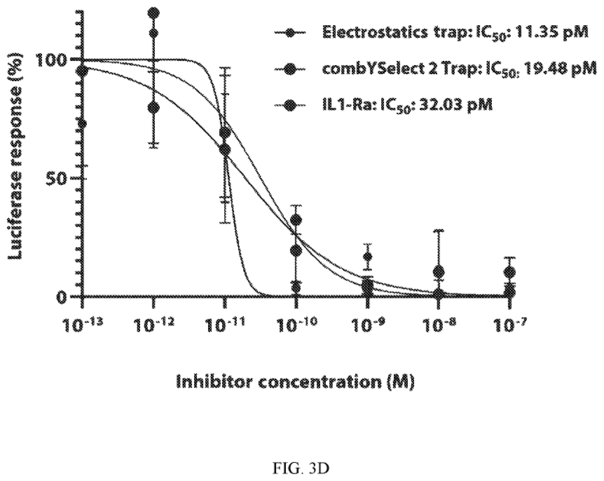

The bispecific antibody was designed with one arm consisting of the rituximab anti-CD20 Fab, while the other was a nanobody specific for HER2 (FIG. 3A). Both of bispecific comb YSelect heterodimers were able to simultaneously bind the two antigens expressed on Raji (CD20+) and BT474 (HER2+) cells, as shown by the formation of cell clusters that were positive for the distinct dyes used to stain each of the two cell types. This was the case regardless of the antigen specificity of each protomer in versions of these bispecific antibodies in which the anti-CD20 Fab and anti-HER2 nanobody were fused to the opposite Fc. Conversely, cell-bridging clusters did not form when using a non-specific IgG1 isotype control or any monospecific heterodimer or homodimer.

IgG heterodimers are also commonly used in Fc-fusion based therapeutics. Accordingly, in a second potential therapeutic scenario, the combYselect2 Fc was used to construct a cytokine trap composed of the IL-1 cytokine cognate receptor IL-1 receptor I (IL-1RI) and secondary receptor IL-1 receptor accessory protein (IL-1RAcP) to sequester IL-1β (FIG. 3C). The ability of the combYSelect2 cytokine trap to sequester IL-1β was measured via inhibition of luciferase expression driven by the IL-8 promoter in HEK293T cells, which express IL-1RI and IL-1RAcP endogenously. Inhibition by the combYSelect2 trap was comparable to that of a trap fused to an electrostatic heterodimer E357Q/S364K-L368D/K370S. Both traps had slightly improved inhibition compared to IL-1 receptor antagonist (IL-1Ra; FIG. 3D), the natural antagonist of IL-1 cytokine signaling, the recombinant form of which is Anakinra that is used to treat rheumatoid arthritis and other chronic inflammatory conditions.

The comb YSelect method is useful to redesign the interface between the CH3 domains on either protomer in the IgG1 Fc homodimer, with an eye toward developing novel heterodimeric Fc regions that could serve as platforms for bispecific antibodies (bsAbs). Commonly, bsAbs are engineered to simultaneously target T-cell specific antigens and tumor associated antigens (TAAs), bridging the two cell types together and enhancing the immune response to tumor cells.

Another common use of bsAbs is for the targeting of two different TAAs simultaneously, which improves specificity and decreases the likelihood of tumors escaping by downregulating specific epitopes or reverting to redundant pathways.

In certain embodiments, this disclosure contemplates IgG-like bsAbs, which retain long serum half-lives due to physiological binding to FcRn, compared to non IgG-like bsAbs that lack the Fc region. It is contemplated that comb YSelect promotes specific associations between a light chain and its correct heavy chain pair allowing for the production of a bsAb that most closely resembles the functionality, stability, and long half-life of a wildtype antibody while retaining the advantages of dual antigen specificity.

It is contemplated that combYSelect antibodies disclosed herein can be utilized as a platform for designing bsAbs of other antibody isotypes, considering that conformations and homodimeric interfaces of the Fcs of other antibody isotypes are similar to IgG Fcs, e.g., using alternative isotypes such as IgA and IgE. Protein and glyco-engineering efforts have improved the producibility, stability, and half-life of IgA, as monomeric IgA has shown efficacy in pre-clinical models. IgA has unique properties that include its ability to engage neutrophils, which highly express FcαRI, making it potentially effective as an anti-tumor therapeutic. Additionally, IgA displays increased stability in mucosal surfaces compared to IgGs, improving potential for mAbs directed at lung- or gastrointestinal-associated inflammation. Similarly, monoclonal IgE binds with extremely high affinity to FcεRI, which is expressed on tumor-associated macrophages, and can improve molecular allergy diagnostics. Thus, engineering bispecific IgA and IgE antibodies contribute to improving the clinical efficacy of such mAbs.

In certain embodiments, this disclosure contemplates a heterodimeric antibody containing heavy chain mutation patterns disclosed herein, e.g., a bispecific antibody. In certain embodiments, the bispecific antibody specifically binds a T-cell specific antigen. In certain embodiments, the T-cell specific antigen is CD3, CD4, CD8, CXCR3, CCR4, CD4, CD25, CD127, and/or CD152.

In certain embodiments, the bispecific antibody specifically binds a tumor associated antigen (TAA). In certain embodiments, the TAA is cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc—O-Ser/Thr), sialyl-Tn (aNeuAc2,6-aGalNAc—O-Ser/Thr), TF (bGal1,3-aGalNAc—O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen, BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-E6 (human papillomavirus E6 protein), MARTI (melan-A), NY-ESO-1 (cancer/testis antigen 1B), PD-L1 (CD274 molecule), PSMA (folate hydrolase 1), or VEGFR2 (kinase insert domain receptor/vascular endothelial growth factor receptor 2).

In certain embodiments, this disclosure contemplates methods of treating a disease or condition with a heterodimeric antibody containing heavy chain mutation patterns disclosed herein by administering an effective amount of the heterodimeric antibody to a subject in need thereof. In certain embodiments, the subject is at risk of, exhibiting symptoms of or diagnosed with cancer, an autoimmune disorder, cardiovascular disease, diabetes, respiratory disease, or inflammatory disease.

1. A recombinant antibody having a heavy chain with mutations K409S and T411Y.

2. The recombinant antibody of claim 1 having a second heavy chain with mutations L368S and D399Y

3. The recombinant antibody of claim 1 having a second heavy chain with mutations D399Y and K447S.

4. A recombinant antibody having a heavy chain with mutations L368S and D399Y.

5. The recombinant antibody of claim 4 having a second heavy chain with mutations K409S and T411Y.

6. A recombinant antibody having a heavy chain with mutations D399Y and K447S.

7. The recombinant antibody of claim 6 having a second heavy chain with mutations K409S and T411Y.

8. A heterodimeric antibody comprising a first heavy chain with mutations K409S and T411Y and a second heavy chain.

9. The heterodimeric antibody of claim 8, wherein the second heavy chain comprising mutations L368S and D399Y.

10. The heterodimeric antibody of claim 8, wherein the second heavy chain comprising mutations D399Y and K447S.

11. A nucleic acid encoding the recombinant heavy chains as in any of claim 1 in operable combination with a heterologous promoter.

12. A vector comprising a nucleic acid of claim 11.

13. A cell comprising a nucleic acid of claim 11 or a vector of claim 12.

14. A heterodimeric antibody comprising a recombinant antibody heavy chain(s) as in claim 8 any of claims 8-10.

15. The heterodimeric antibody of claim 14 which is a bispecific antibody.

16. The bispecific antibody of claim 15 which specifically binds a T-cell specific antigen.

17. The bispecific antibody of claim 15 which specifically binds a tumor associated antigen.


SEQUENCE LISTING (1 sequence)

SEQ ID NO: 1 - protein, 329 aa, synthetic construct
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK